(19) United States
(12) Patent Application Publication
(10) Pub. No.: US 2005/0019261 A1
(43) Pub. Date: Jan. 27, 2005

(54) Screening method for compounds that modulate neuronal activity

(76) Inventors: Hugo Albrecht, Bottmingen (CH); Mirna Kvajo, Basel (CH); Denis Monard, Fullinsdorf (CH)

Correspondence Address: NOVARTIS;CORPORATE INTELLECTUAL PROPERTY, ONE HEALTH PLAZA 430/2, EAST HANOVER, NJ, 07936-1080, US

(21) Appl. No.: 10/497,089

(22) PCT Filed: Nov. 29, 2002
(86) PCT No.: PCT/EP02/13519

(30) Foreign Application Priority Data
Nov. 30, 2001 (GB) 0128793.7

(51) Int. Cl. A61K 49/00; C12Q 1/00; G01N 33/53; G01N 33/567

(52) U.S. Cl. 424/9.2; 435/4; 435/7.2

(57) ABSTRACT

Animal models and screening methods are provided useful for the identification of modulators of neuronal activity and plasticity, as well as for diagnostic purposes. The methods are based on the use of a serine protease inhibitor, in particular PN-1, as an indicator of neuronal activity.

The present invention relates to the fields of neurobiology, pharmacology and medicine. More particularly, the invention relates to screening methods for identifying agonists and antagonists of a neuronal circuit. Such compounds are useful for the treatment of behavioural and learning disorders, as well as to monitor the consequences of neurodegenerative diseases. The invention provides a way to monitor activity in neuronal circuits following specific behaviour paradigms and to identify compounds specifically regulating such circuits.

Localized proteolytic activity through the action of proteases plays a critical regulatory role in a variety of important biological processes. A wide variety of ideas about the function of serine proteases and their inhibitors in embryonic development and in the nervous system have been proposed. It has been suggested that serine proteases, such as plasminogen activators or thrombin, are involved in restructuring of the synaptic connectivity during development and regeneration. Such processes include elimination during development and synaptic plasticity associated with learning and memory in the adult. The balance of proteases and their respective biological inhibitors may provide regulation of these complex biological processes.

One of the best-studied endogenous serine protease inhibitors is protease nexin-1 (PN-1). PN-1 is a 43 kDa serine protease inhibitor of the serpin superfamily (Sommer et al., Biochemistry, 1987; 26(20):6407-10). The serpin superfamily includes neuroserpin, brain-associated inhibitor of tissue-type plasminogen activator (BAIT), as well as protease nexin-1 (PN-1). See, for instance, Osterwalder, T., et al., EMBO J 1996; 15:2944-2953, or U.S. Pat. No. 6,008,020.

In vitro, PN-1 inhibits several serine proteases including thrombin, trypsin, tissue-plasminogen activator and urokinase-plasminogen activator (Guenther, et al., EMBO J. 1985;4(8):1963-6). In vivo, PN-1 is expressed in several organs during embryonic development, including cartilage, lung, skin, urogenital tract, and the central and peripheral nervous systems with a complex spatial and temporal expression pattern in the developing central nervous system (Mansuy, et al., Development 1993;119(4):1119-34). In the mature central nervous system (CNS), PN-1 is constitutively expressed in specific neuronal populations such as the olfactory nerve layer of the olfactory bulb, the pyramidal neurons of layer V in the cortex and the inferior and superior collicull (Reinhard et al., J Neurosci Res., 1994; 37(2):256-70). Nevertheless, PN-1's in vivo partner(s) and physiological function remain elusive.

Mice overexpressing PN-1 and PN-1 knockout mice have shown to be seizure prone and to develop epileptic activity. Theta burst-induced long-term potentiation (LTP) and NMDA (N-methyl-D-asparate) receptor-mediated synaptic transmission in the CA1 field of hippocampal slices were augmented in PN-1 overexpressing mice and reduced in PN-1 knock-out mice. Changes in GABA-mediated inhibition in PN-1 overexpressing mice suggested that altered brain PN-1 levels lead to an imbalance between excitatory and inhibitory synaptic transmission (Luthi et al., J Neurosci. 1997;17(12):4688-99).

It has been reported that PN-1 is transiently upregulated in rat sciatic nerve after axotomy, and PN-1-producing cells are localized distal to the lesion site. This up-regulation of PN-1 occurs 2-3 days after a similar up-regulation of prothrombin and thrombin in the distal stump (Meier et al., Nature. 1989;342(6249):548-50). In patients with Alzheimer's disease, free PN-1 protein is significantly decreased, while endogenous PN-1 -thrombin complexes are increased in the brain (Wagner et al., Proc Natl Acad Sci USA, 1989; 86(21):8284-8). PN-1 is upregulated in the putamen following 6-hydroxydopamine-induced cell death in the substantia nigra, a model of Parkinson's disease (Scotti et al., J Neurosci Res. 1994;37(2):155-68). It has also been reported that PN-1 rescues spinal motoneuron death in the neonatal mouse. (Houenou, L. J. et al., Proc Natl Acad Sci USA. 1995;92(3):895-9).

Recent studies suggest that neuronal plasticity is involved in pathways such as pain pathways, (Anton et al., Neuroscience 1991;41(2-3):629-41; or Mineta et al. Exp Brain Res 1995; 107(1): 34-38), fear avoidance pathways, social stress pathways (Kollack-Walker et al., J Neurosci. 1997;17(22):8842-55) and vibrissae pathways (Barth et al, J Neurosci. 2000;20(11):4206-16; or Staiger et al., Neuroscience. 2000;99(1):7-16).

There is a need for screening methods for compounds that specifically modify the activity of neuronal circuits. In particular, there remains a need for an in vivo model to test for neuronal activity and plasticity of neuronal circuits, as well as testing for the physiological function of both novel and well-known factors that regulate neuronal activity and this invention meets that need. In addition, there is a need to identify compounds that will influence the expression of serine protease inhibitors in vivo and therefore also play a role in the regulation of serine proteases, particularly in the nervous system, since disturbances of such regulation may be involved in disorders relating to haemostasis, anglogenesis, tumour metastasis, cellular migration, as well as neurogenesis; and therefore, there is a need for identification and characterization of candidate compounds which can play a role in preventing, ameliorating or correcting such disorders.

The present invention provides a method to anatomically identify and monitor any neuronal circuit activated in vivo. In particular, this invention provides a method of screening compounds that modulate neuronal activity comprising contacting brain tissue in an animal with a candidate compound; assaying for a change in serine protease inhibitor expression levels, in particular, protease nexin-1 (PN-1) expression levels, relative to when the candidate compound is absent, wherein the PN-1 expression in a particular brain region is indicative of a disease, disorder or condition of interest; and selecting a compound that modifies the serine protease inhibitor expression level, in particular PN-1 expression level, relative to when the compound is absent as a modulator of neuronal activity and/or changes in neuronal plasticity.

The candidate compound may reduce or increase the expression level of the serine protease inhibitor, in particular PN-1. A compound that decreases neuronal activity is a potential modulator of conditions where neuronal activity is increased, such as in epilepsy, drug abuse, positive psychotic symptoms or hyperactivity syndromes. A compound that increases neuronal activity is a potential modulator of conditions where neuronal activity is decreased, such as in neurodegenerative disorders, dementias of the Alzheimer or Non-Alzheimer type, Parkinson's disease, vascular strokes, benign and malign tumours or injuries affecting the cortex of the brain.

In a further aspect of the invention, the screening method further comprises inducing neuronal activity in a specific region of the brain, for example and without limitation by influencing behaviour or administering a chemical agent (e.g., 6-hydroxydopamine, ibotenic acid, or any neurotoxic agent).

In a preferred embodiment of the invention, the PN-1 expression level is detected through the presence of a protein marker expressed under the control of PN-1 gene regulatory sequences (PN-1 promoter sequences), preferably a protein marker encoded by a reporter gene, more preferably a reporter gene selected from the group consisting of β-galactosidase, glucosidases, chloramphenicol acetyltransferase (CAT), glucoronidases, luciferase, peroxidases, phosphatases, oxidoreductases, dehydrogenases, transferases, isomerases, kinases, reductases, deaminases, catalases, urease, and fluorescent proteins.

In an alternative embodiment, the PN-1 expression level is detected through the presence of a peptide or protein tag, such as hemagglutin (HA), Glutathione S-transferase (GST), His6, or myc.

Therefore, the invention provides a method for screening compounds that potentially modulate neuronal activity or changes in neuronal plasticity by using expression of a serine protease inhibitor, preferably a protease nexin-1 like protein, more preferably PN-1, as an indicator for neuronal activity in a specific region of the brain.

The present inventors have prepared stem cells and progenitor cells expressing PN-1, in particular in the brain, which are also encompassed by the invention. Also provided is a method of selecting stem cells or progenitor cells characterized by having PN-1 expression above basal levels (i.e., PN-1 promoter activity) from a mixed population of cells comprising sorting cells carrying a protein detectable by fluorescence from the remaining cells, wherein the protein is expressed under the control of PN-1 promoter sequences.

The present invention also provides non-human animal models for the study of neuronal activity and/or neuronal plasticity based on expression levels of PN-1, for the screening of candidate pharmaceutical compounds or the evaluation of potential therapeutic interventions, and for the creation of explanted mammalian cell cultures which express a protein marker (e.g., reporter gene) under the control of PN-1 regulatory sequences. PN-1 regulatory sequences may be present as the complete PN-1 gene or as a fragment thereof comprising a promoter and enhancer elements as is well known in the art, preferably as the complete PN-1 gene, as in the form of a knock-in animal. Also provided are cells extracted from the knock-in animals, their progeny or equivalents and their use in in vitro screening assays for modulators of neuronal activity and/or neuronal plasticity.

The present invention further provides an in vivo screening method for identifying compounds in a fast and reliable way, within which a serine protease inhibitor is used to anatomically identify and monitor neuronal circuits associated with behaviour and neuronal activity in vivo, independent of whatever genes that might be involved in the neuronal circuit. The method advantageously allows quantification of neuronal activity, for example, after modulation by a candidate compound. The present invention provides an indicator or readout for processes resulting in neuronal activity and can be applied in a fast and reliable in vivo behaviour assay. Compared to electrophysiology, one of the best known methods to monitor neuronal activity such as epilepsy (see for example Tatum WO IV, J Clin Neurophysiol. 2001;18(5):442-455), the present invention provides a method for quantitatively identifying structures in the brain which are actually involved in neuronal activity elicited by behaviour, diseases paradigms or other conditions of interest. The present invention provides a screening method that is much faster than electrophysiology and which optionally can be used in conjunction with electrophysiological methods.

The present invention provides a way to monitor neuronal activity in vivo and to show in an in vivo behaviour model that serine protease inhibitor expression is regulated by neuronal activity. As a result, screening methods for identifying candidate compounds capable of acting as agonists or antagonists of the expression of serine protease inhibitors are provided. For the first time, the present inventors have shown in animals that PN-1 expression is upregulated upon neuronal activity.

In addition, little is known at present about whether the regulation of gene transcription is necessary for experience-dependent plasticity. Only if this question is answered, can diagnostic methods for detecting disorders related to the central and peripheral nervous system and the circulatory system, and therapeutic methods for treating such disorders be facilitated. To answer this question and to be able to provide compounds used to diagnose and treat disorders, and to study the pathways involved in neocortical plasticity, a reliable in vivo system is required in which the cellular locus of plasticity can be clearly identified.

The present invention is useful for the diagnosis of various nervous system-related disorders in mammals, including humans. Such disorders include impaired processes of learning and memory, including impaired spatial, olfactory and taste-aversion learning, learning and memory impairments associated with Alzheimer's, and impairments associated with other degenerative diseases, such as Parkinson's disease and the like. The present invention provides a method to anatomically identify and monitor any neuronal circuit activated in vivo and those neuronal circuits that might be impaired by detecting expression of a serine protease inhibitor, in particular PN-1. Thus, a particular pattern of PN-1 expression can be associated with a given disease, disorder or condition of interest and used to diagnose the same.

Also provided is a method of screening compounds that modulate neuronal activity comprising contacting brain tissue in an animal with a candidate compound, assaying for a change in serine protease inhibitor expression, preferably a change in the expression level of a protease-nexin-1 like protein (e.g., neuroserpin, BAIT or other member of the serpin superfamily), most preferably a change in protease nexin-1 (PN-1) expression level relative to when the candidate compound is absent, wherein the serine protease inhibitor expression in a particular brain region is indicative of a disease, disorder or condition of interest; and selecting a compound that modifies the serine protease inhibitor expression level relative to when the compound is absent, as a modulator of neuronal activity and/or plasticity.

The term “modulate” for the purpose of the present invention shall mean changing the level of serine protease inhibitor expression, preferably PN-1 expression, in the brain, either reducing or increasing such level, for example relative to a control.

The term “neuronal activity” for the purpose of the present invention includes activity of neuronal circuits triggered by for example behaviour, pain, leaming, fear, stress, or drugs, such activity being involved in passing an input through different neuronal structures within a circuit to process an input, as exemplified by the vibrissae pathway, where the response of the neuronal circuit is involved in the transfer of sensory information from the level of the whiskers to the level of the cortex.

The term “neuronal plasticity” for the purpose of the present invention includes changes in the efficiency of neuronal circuits caused by behaviour, pain, fear, lesions, or neurodegenerative diseases. Each disorder or disease will have a specific circuit that will be affected.

The term “proteolytic activity” for the purpose of the present invention includes enzymatic activity leading to the activation of protease-activated receptors or to the degradation of different proteins in the cellular microenvironment, including components of the extracellular matrix.

The term “contacting” for the purpose of the present invention includes administering the candidate compound by any method known in the art, for example, orally, or by intravenous (preferably in the tail vein), intramuscular, intraperitoneal, intrastemal, subcutaneous and intraarticular injecuon and infusion. Although the candidate compound may induce a desired effect in the brain, typically the candidate compound will act directly in the brain or specific region thereof and therefore the ability of the candidate compound to cross the blood-brain barrier must be considered as is well known in the art. Preferably, the compound is administered orally, provided that it can cross the blood-brain barrier.

The term “candidate compound” for the purpose of the present invention includes any compound that can change the expression level of a serine protease inhibitor, such as PN-1 in the brain, and preferably that is able to cross the blood-brain barrier in the brain. Alternatively, in some embodiments, the candidate compound can be modified after identification to cross the blood-brain barrier. The candidate compound can either act as an antagonist or agonist of the expression of a serine protease inhibitor. Potential antagonists in the present invention include small organic molecules, peptides, polypeptides and antibodies that bind to a polypeptide of the invention and thereby inhibit or extinguish its activity, but for ease of administration are preferably small organic molecules. Potential agonists can also include small organic molecules, peptides and polypeptides.

Other potential antagonists include antisense or siRNA (small interfering RNA) molecules. Antisense technology can be used to control gene expression through antisense DNA, or RNA or through triple-helix formation. Antisense techniques are discussed, for example, in Okano, J. Neurochem., 1991;56:560. The methods are based on binding of a polynucleotide to a complementary DNA or RNA. For example, the 5′ coding portion of a polynucleotide that encodes the mature polypeptide of the present invention may be used to design an antisense RNA oligonucleotide of from about 10 to 40 base pairs in length. A DNA oligonucleotide or siRNA is typically designed to be complementary to a region of the gene involved in transcription thereby preventing transcription and the production of PN-1. The antisense RNA oligonucleotide hybridises to the mRNA in vivo and blocks translation of the mRNA molecule into PN-1 polypeptide. The oligonucleotides described above can also be delivered to cells such that the RNA or DNA may be expressed in vivo to inhibit production of PN-1. The antagonists of PN-1 expression (or any other serine protease inhibitor antagonist) may be used in a method for treating an individual in need of a decreased level of PN-1 activity in the body (i.e., less inhibition of a protease susceptible to PN-1) comprising administering to such an individual a composition comprising a therapeutically effective amount of an antagonist of PN-1 expression.

The term “animal” for the purpose of the present invention includes any kind of mammals with the exception of humans. Animal species which are suitable for use in the animal models of the present invention include, but are not limited to, rats, mice, hamsters, guinea pigs, rabbits, dogs, cats, goats, sheep, pigs, and non-human primates such as monkeys and chimpanzees. For initial studies, transgenic mice and rats are highly desirable due to their relative ease of maintenance and shorter life spans. For certain studies, transgenic yeast or invertebrates may be suitable and preferred because they allow for rapid screening and are easier to handle. For long-term studies, non-human primates may be desired.

The present invention provides non-human animal models for the study of neuronal activity and/or neuronal plasticity based on expression levels of PN-1, for the screening of candidate pharmaceutical compounds or the evaluation of potential therapeutic interventions, and for the creation of explanted mammalian cell cultures which express a protein marker (e.g., reporter gene product) under the control of PN-1 regulatory sequences. PN-1 regulatory sequences may be present as the complete PN-1 gene or as an operable fragment thereof, preferably as the complete PN-1 gene, as in the form of a “knock-in” animal.

Prior to the present invention, no animal model was available where PN-1 expression had been linked to neuronal activity. Although a PN-1 transgenic mouse has been reported (incomplete 3,7 kb promoter, see for example Kury et al., Development. 1997;124(6):1251-62; or Mihailescu et al., Mech Dev. 1999;84(1-2):55-67), PN-1 expression was visualized mainly in the embryonic mid/hind brain junction and does not suggest PN-1 expression as an indicator of neuronal activity or changes in neuronal plasticity. Only after generating the PN-1 knock-in mice did the present inventors unexpectedly find that PN-1 expression could indeed be used as such an indicator. Increased PN-1 expression is demonstrated in response to behaviour (such as social stress, pain, an the like. See examples below). The animal preferably comprises a marker protein (e.g., reporter gene) under the control of PN-1 regulatory sequences.

A “protein marker” as used herein is a gene product that can be used as a signal for the expression of a gene such as a serine protease inhibitor gene and preferably to quantitatively measure the expression pattern in the brain upon neuronal activity. Examples for such protein markers can be any detectable protein (e.g. using an antibody specific for the protein being expressed), such as PN-1, or proteins expressed by reporter genes including without limitation β-galactosidase, glucosidases, chloramphenicol, acetyltransferase (CAT), glucoronidases, luciferase, peroxidases, phosphatases, oxidoreductases, dehydrogenases, transferases, isomerases, kinases, reductases, deaminases, catalases, urease and fluorescent proteins, such as green fluorescent protein. In a more preferred embodiment the present invention employs an enzymatic marker, such as β-galactosidase as the marker protein.

An “operable fragment” of the PN-1 gene in the sense of the present invention means any fragment of the PN-1 gene that can drive the expression of a sequence operably linked thereto.

A serine protease inhibitor, preferably a protease nexin-1 like protein, can be used as an indicator for neuronal activity in a specific region of the brain. The screening methods provided herein allow the identification of agonists and antagonists of serine protease inhibitor expression in vivo, as well as compounds for medical and non-medical uses. In a preferred embodiment of this invention the serine protease inhibitor is PN-1. Although this invention is exemplified using PN-1 as a serine protease inhibitor, any other PN-1 like protein can be used which includes, but is not limited to, BAIT or neuroserpin. In this assay, an increase in the reporter compared to the standard indicates that the candidate compound enhances PN-1 expression and a decrease in the reporter compared to the standard indicates that the compound antagonizes PN-1 expression.

There are several ways in which to create an animal model for neuronal activity/plasticity, one of which is exemplified in Example 1 below. In a preferred embodiment knock-in mice or rats are generated with an insertion of an internal-ribosomal-entry-site/reporter construct at the 3′-end of the serine protease inhibitor locus. Although the examples below illustrate the invention using β-galactosidase, it will be apparent to one of ordinary skill in the art in light of the specification that other reporter proteins can be employed, in the presence or absence of the endogenous PN-1 gene sequences. The insertion of recombination elements, such as lox p sites, recognized by enzymes such as Cre recombinase can also be used to advantage. To create a mouse, the desired nucleic acid can be inserted into a mouse germ line using standard techniques of oocyte microinjection or transfection or microinjection into embryonic stem cells followed by homologous recombination in embryonic stem cells. In general, techniques of generating transgenic/knock-in animals are widely accepted and practiced. A laboratory manual on the manipulation of the mouse embryo, for example, is available detailing standard laboratory techniques for the production of transgenic mice (Hogan et al., 1986).

In a further aspect of the present invention the role of serine protease inhibitors in synaptic transmission and plasticity and the influence of neuronal activity on the expression of these serine protease inhibitors is assessed in vivo by using knock-in animals. Preferably, the methods further comprise administering the test agent to a control animal in which neuronal activity is not induced, so that the effect is determined by reference to a control.

In one embodiment, the present invention provides a system to anatomically identify and monitor any neuronal pathway activated in vivo. “Neuronal pathway” for the purpose of the present invention includes the group of neurons building a circuit triggered by sensory events, such as for example behaviour, pain, fear, lesion or diseases. The vibrissae pathway including the barrel cortex has been chosen to exemplify the invention (see examples below) because of the functional and anatomical correlation between the vibrissae and the barrels within the barrel cortex. The connections in this system start with the whisker-sensitive receptor cells in the whisker follicle, from which the signals travel to the trigeminal brainstem nuclear (TBNC) complex. Several nucleae are innervated: the trigeminal nucleus principalis (PrV) and the three subnuclei of the spinal trigeminal nucleus: oralis (SpVo), interpolaris (SpVi) and caudalis (SpVc). Axons from the TBNC then project to portions of the contralateral thalamus, and from there to the barrels in layer IV of the somatosensory cortex. Therefore, the clearly defined anatomical map of the sensory whisker pad (Woolsey et al., Brain Res. 1970;17(2):205-42) can be used to show whether changes in expression of a specific gene are specific to changes in experience through particular whiskers, and whether neuronal activity regulates expression of such a gene.

Although exemplified by the vibrissae pathway, the invention has applications in any other neuronal pathway of interest, for example in Parkinson's disease, behaviour, pain, fear, lesion, stress, drug addiction, diseases, or other sensory events. Other examples of neuronal circuits have been described in details in Paxinos, G. (Ed.), “The Rat Nervous System”, Second Edition, Academic Press, San Diego, 1995.

In a further embodiment of the present invention neuronal activity is induced in a specific region of the brain, for example, by behaviour, by chemical induction, by physical induction, or through environmental constraints. “Behaviour” as used herein includes leaming, habituation, or reaction to stress, pain, fear, anxiety, or drugs. Examples of a chemical agent that enhances or decreases neuronal activity includes without limitation 6-hydroxydopamine, ibotenic acid, Ab amyloid peptid, kainic acid, isoniazid, nitrozepan, benzodiazepine, phenylenetetrazole, N-methyl-D-aspartate, quisqualate, picrotoxin, bicuculin, or any other neurotoxic agent.

The present invention also relates to pharmaceutical preparations wherein the active agent is a compound of the invention. Therefore, the present invention provides methods for treating an individual with a disease, disorder or condition of interest with a compound identified by the methods of the invention. The active agent can be mixed with excipients that are pharmaceutically acceptable and compatible with the active agent and in amounts suitable for use in the therapeutic methods described herein. Suitable excipients are, for example, water, saline, dextrose, glycerol, ethanol or the like and combinations thereof, including vegetable oils, propylene glycol, polyethylene glycol and benzyl alcohol (for injection or liquid preparations); and petrolatum, vegetable oil, animal fat and polyethylene glycol (for externally applicable preparations). In addition, if desired, the composition can contain wetting or emulsifying agents, isotonic agent, dissolution promoting agents, stabilizers, colorants, antiseptic agents, soothing agents and the like additives (as usual auxiliary additives to pharmaceutical preparations), pH buffering agents and the like which enhance the effectiveness of the active ingredient.

The therapeutic compositions of the present invention can include pharmaceutically acceptable salts of the components therein. Pharmaceutically acceptable salts include the acid addition salts (e.g. formed with free amino groups) that are formed with inorganic acids such as, for example, hydrochloric or phosphoric acids, or such organic acids as acetic, tartaric, mandelic and the like. Salts formed with the free carboxyl groups can also be derived from inorganic bases such as, for example, sodium, potassium, ammonium, calcium, or ferric hydroxides, and such organic bases as isopropylamine, trimethylamine, 2-ethylamino ethanol, histidine, procaine and the like.

As used herein, the terms “pharmaceutically acceptable”, “physiologically tolerable” and grammatical variations thereof, as they refer to compositions, carriers, diluents and reagents, are used interchangeably and represent that the materials are capable of administration to or upon a subject, e.g., a mammal, without the production of undesirable physiological effects such as nausea, dizziness, gastric upset and the like.

Physiologically tolerable carriers are well known in the art. Exemplary of liquid carriers are sterile aqueous solutions that contain no materials in addition to the active ingredients and water, or contain a buffer such as sodium phosphate at physiological pH value, physiological saline or both, such as phosphate-buffered saline. Still further, aqueous carriers can contain more than one buffer salt, as well as salts such as sodium and potassium chlorides, dextrose, polyethylene glycol and other solutes. Liquid compositions can also contain liquid phases in addition to and to the exclusion of water. Exemplary of such additional liquid phases are glycerine, vegetable oils such as cottonseed oil, and water-oil emulsions.

Further included in this invention is a method of specifically increasing neuronal activity in the brain with an effective amount of such a compound, as well as a method of specifically decreasing neuronal activity in the brain with an effective amount of such a compound. Such a method may be employed for medical as well as non-medical purposes. For example, a method of treatment of a disease displaying increased neuronal activity with an effective amount of a compound according to this invention that decreases neuronal activity is included in this invention. Examples of such diseases are epilepsy, drug abuse, positive psychotic symptoms or hyperactivity syndromes. In addition, a method of treatment of a disease displaying decreased neuronal activity with an effective amount of a compound according to this invention that increases neuronal activity is part of this invention. Diseases displaying decreased neuronal activity are numerous, e.g. dementias of the Alzheimer or Non-Alzheimer type, Parkinson's disease, vascular strokes, benign and malign tumours or injuries affecting the cortex of the brain. Every disease that affects the human brain may also lead to a modulation of neuronal activity in the sense of this invention. The term “condition of interest dependent on neuronal activity” as used in the present invention includes any disorder, such as, but not limited to, learning disorder, specific behaviour disorder and the like but also drug or alcohol abuse, obesity, fear, stress or other conditions that are not desirable but are not necessarily considered as disorders or diseases.

Thus, non-medical purposes of such compounds are also contemplated by this invention. Examples are compounds of the invention that increase neuronal activity associated with behaviour in a mild to moderate fashion and may be consumed on a daily basis, i.e. for the enhancement of performance. The term “performance” for the purposes of this invention includes e.g. the learning and the memory functions of the brain.

The present inventors have observed stem cells and progenitor cells expressing PN-1 and therefore also encompassed by the invention is a method of the invention, wherein PN-1 is expressed in stem cells or progenitor cells in the brain.

Furthermore, also provided is a method of selecting stem cells or progenitor cells expressing a gene product under the control of PN-1 regulatory sequences from other cells is provided, where the gene product is detected by fluorescence. The gene product itself can be fluorescent (e.g. green fluorescent protein (GFP)) or may be detected by an enzymatic reaction releasing a fluorescent molecule. The cells are characterized by having PN-1 expression above basal levels (i.e., PN-1 promoter activity) and are preferably selected from a mixed population of cells using the fluorescence-activated cell sorter (FACS). The selected cells therefore carry a protein detectable by fluorescence, which is expressed under the control of PN-1 promoter sequences, optionally together with PN-1. In one embodiment, cells expressing a reporter gene, as exemplified by β-galactosidase, can be purified by a method that utilizes a fluorescent substrate for the reporter gene in conjunction with a FACS (see for example Abe et al., Dev Biol. 1996; 180(2):468-72). The sorted cells are useful for in vitro screens, such as in screens identifying modulators of neuronal activity or plasticity.

Although the description above refers to the use of the animal models of the invention for the identification of agents that modulate neuronal activity or changes in neuronal plasticity in vivo, the invention is not so limited. In accordance with another aspect of the invention, explanted mammalian cell cultures that express PN-1 or a protein marker (e.g., reporter gene) under the control of PN-1 regulatory sequences can be used to screen for agents that modulate neuronal activity or changes in neuronal plasticity.

Thus, the invention provides a method of identifying an agent that modulates neuronal activity or changes in neuronal plasticity, comprising administering a test agent to a PN-1 knock-in animal of the invention subject to a specific pattern of neuronal activity. Further information will be obtained following isolation of one or more of neuronal cells, glia cells, astrocytes, stem cells and progenitor cells, in particular PN-1 expressing cells from the animal, culturing these cells in vitro and determining at least one effect of the test agent on the cells.

The invention also provides a method of identifying an agent that modulates neuronal activity or (changes in) neuronal plasticity, comprising contacting one or more of neuronal cells, glia cells, astrocytes, stem cells and progenitor cells isolated from a PN-1 knock-in animal of the invention (or progeny or equivalent thereof) with a test agent in vitro and determining at least one effect of the test agent on the cells. By “equivalents” thereof it is meant cells that are genetically modified in vitro to have the same attributes as the cells isolated from the knock-in animal, for example, by transfection. In preferred embodiments, the effect of the agent on serine protease inhibitor expression, in particular PN-1 expression, is determined after induction of neuronal activity in vivo or in vitro.

Preferably, the methods of the invention further comprise testing the effect of a test agent on a control, such as control cells or animals in which neuronal activity is not induced, so that any effect is determined by reference to a control.

The invention is further described below, for the purpose of illustration only, in the following examples.

### EXAMPLE 1

### Baseline Levels of PN-1 Expression

In this example, the basal levels of transcription in animals kept under standard housing conditions are examined to determine whether changes in PN-1 expression occur during neuronal activity. 60 μm thick microtome sections were processed for β-galactosidase histochemistry. β-galactosidase histochemistry on sections was performed in the following way: After 24 or 72 hrs of deprivation and/or enriched environment (see example 2 and 3), experimental animals were anaesthetized with Ketarom and decapitated. The brains were rapidly dissected out in ice-cold phosphate buffered saline (PBS) and quickly frozen in Tissue-Tek O.C.T (Sakura Finetek, Europe). The tissue was sectioned on the cryostat to 60 μm thickness and shortly dried on air. Tissue was then subjected to 20 min of fixation in 4% paraformaledhyde (PFA) and then washed 3 times for 15 minutes in solution B (2 mM MgCl2, 0.02% Nonidet P40, 0.01% sodium deoxycholate), and incubated overnight at 37° C. in solution C (solution B plus 5 mM potassium ferricyanide, 5 mM potassium ferrocyanide, and 0.6 mg/ml 5-bromo-4-chloro-3-indolyl-b-D-galactopyranoside (X-gal) (Boehringer Mannheim).

For whole mount staining of brains, the brains were rapidly dissected out into ice-cold PBS and then submerged in ice-cold fixative (4% paraformaldehyde, PBS) for 2 hr on ice. Tissue was then subjected to two 30 min washes at room temperature in PBS, one 30 min wash at room temperature in solution B (PBS, 2 mM MgCl2, 0.02% Nonidet P40, 0.01% sodium deoxycholate), incubated overnight at 37° C. in solution C (solution B plus 5 mM potassium ferricyanide, 5 mM potassium ferrocyanide, and 0.6 mg/ml 5-bromo-4-chloro-3-indolyl-b-D-galactopyranoside (X-gal) (Boehringer Mannheim).

The activity of the β-galactosidase in brain homogenates was measured with the chemiluminescent reporter gene assay system Galacto-Star according to the protocol of the supplier (Tropix, Bedford, Mass.) (explained below). Samples were prepared in the following way: Animals were intraperitoneally anaesthetized with Ketarom (Franks and Lieb, 1994, Nature, 367:607-614) and perfused with ice-cold PBS. Whole brains were quickly dissected and homogenized in a lysis solution provided by the assay system supplier (100 mM potassium phosphate (pH 7.8), 0.2% Triton X-100).

The homogenate was centrifuged and the amount of proteins in the supernatant was quantified using Bio-Rad Protein Assay, from Bio-Rad Laboratories, Munich. The protein content was standardized and the supernatant was tested for β-galactosidase activity in a series of dilutions. β-galactosidase activity was assessed with a chemiluminescent substrate and measured with a luminometer.

The basal level of PN-1 expression was detected in several regions of the brain. Several distinct areas show strong β-galactosidase activity. Structures such as the olfactory nerve layer and inferior colliculus are particularly prominent. Parts of the visual system, like the visual cortex and anterior pretectal nucleus show a very strong labelling. β-Galactosidase activity is prominent in some thalamic nucleae, the striatum, and several nucleae of the brain stem, such as the spinal trigeminal nuclei, the lateral reticular nucleus and the facial nucleus. β-galactosidase activity was also seen to be specifically present in specific cell populations such as the layer V of pyramidal neurons in the cortex and the Purkinje cells in the cerebellum. These data show that sensory structures express PN-1 very strongly.

For this experiment PN-1 knock-in animals were generated similar to the PN-1 knockout mice described in Luthi et al. 1997. In brief, PN-1 knockout mice (Luthi, et al., J Neurosci. 1997 Jun 15;17(12):4688-99) were generated by homologous recombination in embryonic stem cells. Mice were backcrossed and maintained in the C57BL/6 mouse line. To avoid possible background differences, all the PN-1 protein quantification assays and proteolytic assays on wild-type animals were performed on standard C57BL6 male mice of the same age. Genotyping was performed by PCR on DNA from tail biopsies. Experimental mice were typically 3-6 months old. For knockout mice, part of the exon II (99 bp), along with the ATG initiation codon, was deleted, while in knock-in mice the whole exon II was replaced with the knock-in construct. In both cases the target construct was introduced in embryonic stem cells and it replaced the genomic sequence by homologous recombination.

The PN-1 knock-in construct in this experiment contains a secretion signal (56 bp, gene bank accession number: X70296) in front of a hemagglutinin (HA)-tagged mature PN-1 sequence (PN-1 cDNA, see for example Gloor et al., Cell. 1986 Dec 5;47(5):687-93; or Sommer et al., Biochemistry. 1987 Oct 6;26(20):6407-10). The PN-1 sequence is separated by an IRES (internal ribosomal entry site) sequence from the NLS-LacZ-pA sequence which is followed by a TK Neo pA cassette. The construct was inserted by homologous recombination in the exon II of the PN-1 gene.

### EXAMPLE 2

### Unilateral Whisker Deprivation and PN-1 Expression

To address the question of whether PN-1 transcription might be induced by changes in sensory experience, all the whiskers on one side of the muzzle are unilaterally removed and the animals are placed in an enriched environment for 72 hrs. The “single spared whisker” deprivation pattern was imposed essentially as described (Barth et al., J Neurosci. 2000 Jun 1;20(11):4206-16) with the difference that PN-1 knock-in mice were used and the whiskers were trimmed and not pulled out. The “unilateral” deprivation pattern was done in the same way, except that all the whiskers on one side were removed. Control animals were anaesthetized briefly before putting them in the cages, but no whiskers were removed.

Brains were then sectioned and processed for β-galactosidase histochemistry (essentially as described in example 1). A unilateral upregulation of PN-1 is clearly visible in the spinal trigeminal nucleus interpolaris and in the spinal trigeminal nucleus oralis. The upregulation is ipsilateral to the side with intact whiskers. The contralateral side shows basal levels of β-galactosidase transcription. To show the effect of a single-whisker experience on the transcription of PN-1 in the barrel field, mice are unilaterally deprived of all but the D1 whisker for a period of 24 hrs.

Whole brains are then processed for “galactosidase histochemistry. The D1 barrel is macroscopically visible as a blue dot on the cortical surface. To examine whether the thalamic nucleus that projects to the barrel field exhibits PN-1 expression, coronal sections which contain the ventroposterior medial (VPm) nucleus of the thalamus are studied. These results show that a restricted population of cells within the somatosensory pathway upregulates PN-1/lac Z expression under sensory conditions that induce plasticity.

### EXAMPLE 3

### PN-1 Transcription and Protein Levels After Enriched Environment

Based on the data obtained with the brain sections, the levels of PN-1 transcription and PN-1 protein levels in the brain upon housing in the enriched environment are quantified. The enriched environment consists of a big cage fitted with objects of different shapes and textures, such as tunnels, grids, plastic objects, aluminium foil and paper. Water and food was freely accessible. If the animals were housed in the enriched environment for more then 24 hrs, the objects in the environment were replaced or displaced or, daily basis. Control animals were single-housed in standard small cages. Prior to the experiment all the animals were single-housed in standard small cages for at least 3 weeks.

The β-galactosidase activity in whole brain homogenates from animals housed in enriched conditions, exposed to enriched environment for 6, 72 hrs and 8 days, and control animals is measured. The results confirm the findings in brain sections. The levels of β-galactosidase activity are significantly higher in mice kept in enriched housing conditions for 72 hrs as compared to control mice. However, the levels of β-galactosidase activity, seem to return to basal levels within 8 days of housing in the enriched environment. The protein levels of PN-1 are quantified using a PN-1-specific mouse monoclonal antibody on a dot-blot. A series of dilutions reveals that PN-1 protein levels are lower in control animals than in animals housed in the enriched environment. The same effect can be observed after 24 and 72 hours of housing in the enriched environment.

### EXAMPLE 4

### Enriched Environment and the Proteolytic Activity in the Brain

To see whether the enhanced transcription and translation of PN-1 is changing the proteolytic activity in the brain, brain homogenates from enriched and control animals are tested. A broad-spectrum chromogenic substrate S-2288 that is specific for tPA, factor IX and some plasma kallikreins are used. The proteolytic activity in the brains of mice exposed to enriched environment is decreased, as compared to control animals. This lowered proteolytic activity indicates a presence of a serine protease inhibitor that is upregulated by neuronal activity. This result shows that the balance between serine proteases and their inhibitors in the brain is highly dependent on neuronal activity and that PN-1 is a major determinant of proteolytic activity in the brain.

### EXAMPLE 5

### Social Stress and Pain Assay

This example illustrates the correlation between the up-regulation of a serine protease inhibitor and neuronal activity is social stress. Knock-in male animals housed in groups subjected to social stress compared to single-housed animals show a strong upregulation of PN-1 in several parts of the brain in stressed animals. Similarly, mice exposed to stimuli that induce fear are expected to show an up-regulation of PN-1 in the forebrain.

Animals subjected to pain, after pulling out whiskers and strong pinching the vibrissae pad for a time of at least 15 min, showed an unilateral upregulation of PN-1 in the brain stem, in the nuclei which have been described as reacting to pain stimuli (facial nucleus, lateral reticular formation, see for example Anton et al., Neuroscience 1991;41(2-3):629-41; or Mineta et al. Exp Brain Res 1995; 107(1): 34-38).

All publication referred to herein as well as priority application GB 0128793.7 are hereby incorporated by reference as if each is referred to individually.

1. A method of screening compounds that modulate neuronal activity, said method comprising:
a) contacting brain tissue in an animal with a candidate compound;
b) assaying for a change in protease nexin-1 (PN-1) expression level in a neuronal circuit relative to when said candidate compound is absent; and
c) selecting a compound that modifies the PN-1 expression level relative to when said compound is absent as a modulator of neuronal activity.

2. A method according to claim 1, wherein step c) comprises selecting a compound that reduces the expression level of PN-1.

3. A method according to claim 1, wherein step c) comprises selecting a compound that increases the expression level of PN-1.

4. A method according to claim 1, further comprising inducing neuronal activity in a specific region of the brain.

5. A method according to claim 4, wherein neuronal activity is induced by behaviour.

6. A method according to claim 4, wherein neuronal activity is induced by a chemical agent.

7. A method according to claim 6, wherein said agent is 6-hydroxydopamine, ibotenic acid, or any neurotoxic agent.

8. A method according to claim 1, wherein said neuronal activity is increased and said compound is a potential modulator of epilepsy, drug abuse, positive psychotic symptoms or hyperactivity syndromes.

9. A method according to claim 1, wherein said neuronal activity is decreased and said compound is a potential modulator of neurodegenerative disorders, dementias of the Alzheimer or Non-Alzheimer type, Parkinson's disease, vascular strokes, benign and malign tumours or injuries affecting the cortex of the brain.

10. A method according to claim 1, wherein said PN-1 expression level in step b) comprises detecting the presence of a protein marker expressed under the control of PN-1 regulatory sequneces.

11. A method according to claim 10, wherein said protein marker is encoded by a reporter gene, said reporter gene being operably linked to PN-1 gene or an operable fragment thereof.

12. A method according to claim 11, wherein said reporter gene is selected from the group consisting of β-galactosidase, glucosidases, chloramphenicol acetyltransferase (CAT), glucoronidases, luciferase, peroxidases, phosphatases, oxidoreductases, dehydrogenases, transferases, isomerases, kinases, reductases, deaminases, catalases, urease, and fluorescent protein.

13. A method according to claim 10, wherein said PN-1 expression level in step b) comprises detecting the presence of a peptide or protein tag, said tag being operably linked to PN-1 or a fragment thereof.

14. A method according to claim 13, wherein said tag is hemagglutin (HA), Glutathione S-transferase (GST), His6, or myc.

15. A method according to claim 1, wherein PN-1 is expressed in stem cells or progenitor cells in the brain.

16. A method of selecting stem cells or progenitor cells expressing PN-1, said method comprising linking PN-1 to a fluorescent protein and sorting cells carrying the fluorescent protein from cells wherein said fluorescent protein is absent.

17. The use of PN-1 as an indicator for neuronal activity.
